(19) United States
(12) Patent Application Publication
(10) Pub. No.: US 2010/0217339 A1
(43) Pub. Date: Aug. 26, 2010

(54) Carbon nanotube micro-array relay system for providing nerve sitmulation output and sensation input acrodd proximal and distal ends of damaged spinal cord

(76) Inventor: Seth A. Kane, Tenafly, NJ (US)

Correspondence Address: Seth Kane, 9 Greentree Terrace, Tenafly, NJ, 07670, US

(21) Appl. No.: 12/380,047

(22) Filed: Feb. 23, 2009

(51) Int. Cl. A61N 1/05

(52) U.S. Cl. 607/2

(57) ABSTRACT

The invention described herein is a Carbon Nano-tube Micro-electrode Array Relay System for Providing Nerve Stimulation output and Sensation input between Proximal and Distal ends of a damaged Spinal Cord. This device detects the signals coming from either end of a damaged spinal cord, amplifies them, and then stimulates the axons on the opposing side from which they were received. This device is intended to provide self-contained data relay for the goal of restoring function to a severed spinal cord. It is not intended for data output to an external source for analysis. This device is biocompatible and has a modular power system.

### US REFERENCES

5092332 (1992 Mar. 3) Philip Lee, Steroid eluting cuff electrode for peripheral nerve stimulation
4541432 (1985 Sep. 17) Pedro Molina-Negro, Electric nerve stimulator device
20080071321 (2008 Mar. 20) Joseph W. Boggs II, Systems and methods of neuromodulation stimulation for the restoration of sexual function
4969468 (1990 Nov. 13) Charles L. Byers, Electrode array for use in connection with a living body and method of manufacture
7292890 (2007 Nov. 6) Todd K. Whitehurst, Vagus nerve stimulation via unidirectional propagation of action potentials
20080228240 (2008 Sep. 16) David J. Edell, Long term bi-directional axon-electronic communication system

### FOREIGN REFERENCES

WO/2002/087683 (2002 Jul. 11) Ehud Cohen, Actuation and control of limbs through motor nerve stimulation
WO/2007/109228 (2007 Sep. 27) Javed Kahn, Apparatus for microarray binding sensors having biological probe materials using carbon nanotube transistors

### BACKGROUND OF THE INVENTION

The field to which this invention pertains is that of biotechnological implants. More specifically, it relates to the field of neuroprosthetics. Although a relatively new field, many types of devices have been made to detect signals coming from the nervous system (₁). The (eventual) goal of many of these technologies is to repair damaged nerves, or to provide control for prosthetic/electronic devices. Currently, much work is being done using implantable micro-electrode arrays made of various metals, to gather information from or supply information to the nervous system (₂). These devices have been used to allow paralyzed individuals to control computer cursors using only their minds, allow monkeys to control robotic actuator, and well as provide very limited sight data to the blind.

These techniques and devices have however fallen far short of their intended goals for various reasons. The first is that these technologies are in their infancy and their development in many respects is a matter of time. The second reason is that many researchers approach nervous system data as they approach any other data, by collecting and analyzing massive quantities of it before taking any action (empirically). This second reason leads directly to the third; the inclusion of massive computing devices and software systems to record, analyze, and manipulate signals coming from the body into a form that is readable by machines or to perform those functions on incoming data so that it is readable by the nervous system. The fourth issue is signal fidelity and resolution. Current microelectrodes are so large (although still microns in diameter) that each one receives signals from tens of axons if not more, leading to very low signal fidelity. Microelectrode arrays currently use anywhere from 1-100 electrodes, which is very low resolution compared to that of a human body's nervous system. The fifth issue is that even if these devices were implemented successfully in their current forms, there would be little to no safeguards in place to prevent an outside party from tampering with a person's nervous system, due to the affinity toward using computer control systems to regulate these devices.

### BRIEF SUMMARY OF THE INVENTION

The invention described herein is a Nanotube Micro-array Relay System for Providing Nerve Stimulation output and Sensation input between Proximal and Distal ends of a damaged Spinal Cord. The goal of this device is to provide a relay system between ends of a severed spinal cord.

This device is comprised of two opposing microelectrode arrays, each identical although facing in opposite directions, each fabricated micro circuit board containing switches, diodes, capacitors and contact points to allow signals to be passed from one micro-array to another. In order to be a viable spinal cord implant, a channel must be made in the center of the device to allow for the flow of cerebro-spinal fluid. The circuitry must be contained within a biocompatible material to allow for implantation. This sheath should be fabricated longitudinal channels to allow for the flow of cerebrospinal fluid around the spinal cord. At the crests of these channels should be synthetic material that allows the device to be sutured into place.

This device will be able to accept various power sources, that would be constructed modularly and fitted at the time of implantation, to allow for a wide range of power generation alternatives. These devices include internal batteries with induction chargers, internal batteries with internal kinetic chargers, induction coils with external battery packs and power transfer systems, or any other system that could provide an equitable power supply to this device.

This device detects the signals coming from either end of a damaged spinal cord, amplifies them, and then stimulates the axons on the opposing side from which they were received.

This device addresses the problems put forth in the previous section thusly: This device has been developed like a work of art which implements the bleeding edge technologies of the field, which have the potential to far out perform current technologies; hence it is unencumbered by the youth of the field since art is immortal. This device has no data recording or analysis functions, nor does it provide any output to a computer system. The device is self contained in respect to its data processing and has no programming beyond that defined by its hardware components. By using carbon nanotubes as electrodes this device would have signal fidelity far beyond that of current microelectrode arrays (₃). Because of the small size of these electrodes, many can be placed on a single array to allow for relatively high resolution. Since this device has no computer interfaces or data manipulation capabilities it is relatively safe from outside tampering with its information relay functions.

### BRIEF DESCRIPTION OF THE SEVERAL VIEWS OF THE DRAWING

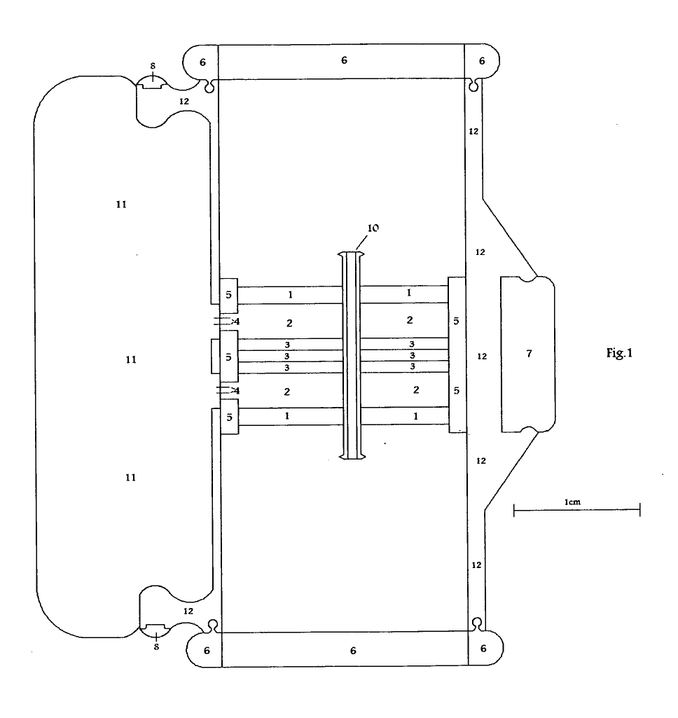

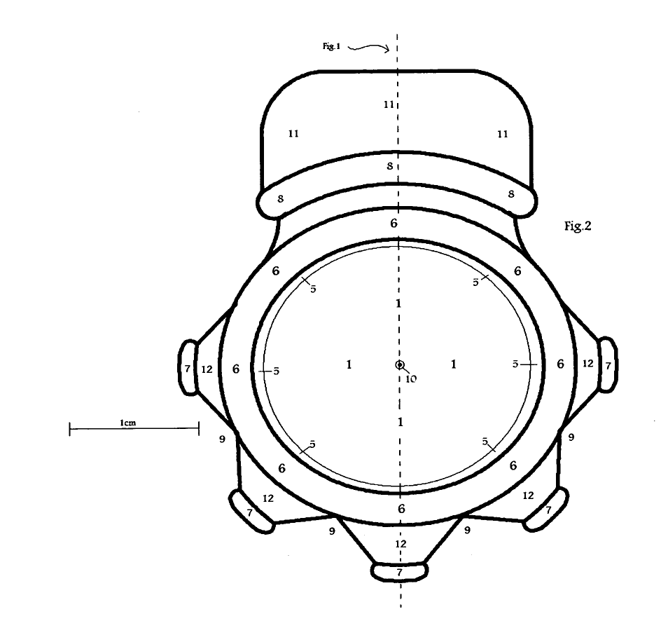

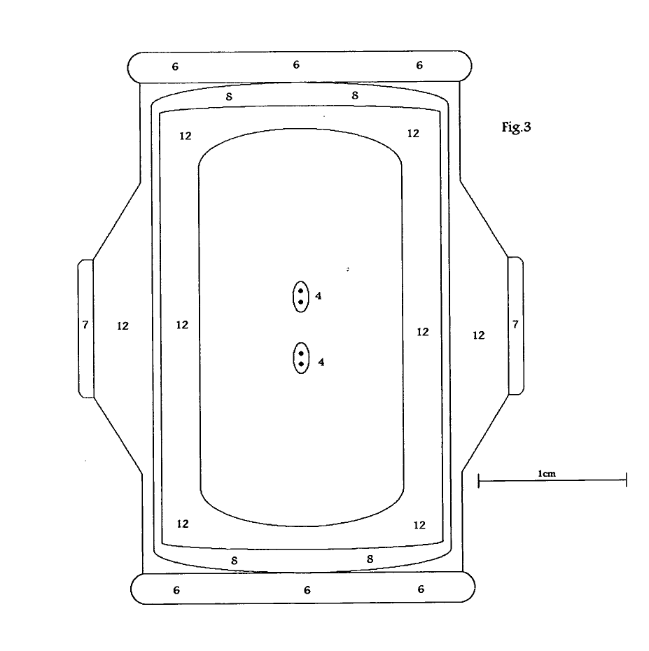

Cross-sectional Side view (FIG. 1). Top down view showing anterior (bottom of page)-posterior (top of page) orientation and plane of FIG. 1 (FIG. 2). Dorsal view (without modular power supply) (FIG. 3).

### DETAILED DESCRIPTION OF THE INVENTION

The device described herein is a Nanotube Micro electrode array Relay System for Providing Nerve Stimulation output and Sensation input between Proximal and Distal ends of a severed Spinal Cord. The main components of this device are: Proximal and distal microelectrode arrays (1) and their subsequent microcircuit boards (2), the inter array junction (3), modular power supply inputs (4), a biocompatible sheath (5), a biocompatible armature (12) with suturable sections (6, 7 and 8) and grooves for cerebrospinal fluid flow (9), a tube to provide fluid flow through the spinal cord's central canal (10), and the modular power supply (11).

The nanotube microelectrode arrays of this device should be made using a combination of standard micro lithography techniques and chemical vapor deposition.

Assuming a minimum microelectrode diameter of 2 micrometers, a maximum electrode density of 5000 electrodes per centimeter could be attained. For the purposes of this device a minimum of 500 electrodes per square centimeter is required. These electrodes should be fabricated directly onto the microcircuit boards (1) that comprise the relay and amplification system. The relay and amplification system (2) functions thusly: When a voltage is generated between a pair of electrodes by a neuronal action potential, a micro switch is activated that closes a circuit which causes a micro capacitor to discharge a voltage of roughly 100 mv (at the electrodes), which passes through the inter array junction, across a pair of electrodes on the opposing surface and then back to the capacitor. Each nanotube electrode has a partner electrode on the same array, across which voltages are detected or discharged, and a mirrored pair on the opposing array to which any signal received is transmitted. Signal transmission in this device is bi-directional however micro diodes should be used to allow for unidirectional signal transmission at any one time. These components should be made using standard micro lithography, chemical vapor deposition and laser etching techniques. The microelectrode array (1) should be assembled on the same silicon dioxide microchip as the capacitor and switching components, however they should be on opposite surfaces of the microchip.

Contacts between the two electrode arrays and subsequent circuit systems should be achieved through the use of an inter array junction (3), into which connections from each array/circuit board pass. There must be a connection for each microelectrode to connect to its mirrored electrode. The junction may have a wider area than the microelectrode array to allow for physical support that may be needed for electrical contacts between opposing arrays. The function of the junction is to allow signals to be transferred between mirrored pairs of electrodes on either array. This junction should be manufactures with vertically aligned nano wires which securely contact nano wires coming from each microarray/auxiliary circuit complex.

Each array (proximal and distal) and its auxiliary components should be identical. These arrays, when placed with their microelectrodes facing away from each other, should be joined together at the inter array junction.

This dual microarray system should be shaped to the cross-sectional dimensions of the spinal cord in the region of the spinal cord at which it would be placed. The preferred embodiment of this device would be such that arrays with different cross-sectional shapes would be made for each region of the spinal cord. These sets of devices with varying cross-sectional shapes would be made in different sizes to allow them to be used on a wide range of individuals with spinal cord damage at varying regions of the cord.

Passing through the center of the arrays, circuits and inter array junction, should be a biocompatible polyethylene tube (10), placed so that it mates on either side of the device with the central canal of the spine. This would allow for an unimpeded flow of cerebrospinal fluid through the device.

Power to this system should be provided through two leads (4) that pass from each circuit board, through the biocompatible housing of the device (to be discussed below), and into ports that allow for the connection of the modular power supply apparatus. Four leads in total are needed, two for each array.

The opposing arrays, microcircuits, and the inter array junction should all be contained within a biocompatible sheath. Said sheath should be made of biocompatible polyethylene (or a comparable material) that is machined or molded using sterile, surgical grade, fabrication techniques. Said sheath should have chamfers that fit around the perimeter of the micro electrode arrays. Said sheath should be flexible enough to be fitted as a single piece around both arrays and auxiliary components and glued into place (using a surgical grade epoxy or “super-glue” to provide a non degradable seal impenetrable to bodily fluids. Said sheath must have sealed ports to allow for power supply leads to pass through it.

Over the sheath a biocompatible armature (12) should be fitted. It should be attached to the sheath using an appropriate adhesive or a mechanical connection. The functions of this armature are multifold. It must be biocompatible (a thicker polyethylene or comparable material is appropriate). It must have channels (9) through which cerebrospinal fluid can pass unimpeded. These channels may be simple longitudinally oriented grooves in the body of the armature to allow for fluid flow. It must have longitudinally oriented regions that allow for suturing (7) and/or tissue in-growth (fabrics such as those used in arterial grafts are appropriate). These regions may be fixed to the body of the armature using a chamfered channel travelling the length of the longitudinal suturing regions (7), along with an appropriate adhesive. These regions are meant to be sutured through the Dura mater of the spinal cord.

The armature must have a suturable region travelling the circumference of the openings into which the proximal and distal ends of the spinal cord pass (6). This suture ring is intended to affix the proximal and distal ends of a severed spinal cord to the device. This suture ring should not impede the flow of cerebrospinal fluid and should not extend to the distance (measured from the central canal duct (10) outward) of the longitudinally oriented suturing regions. These regions are intended to be sutured directly to the spinal cord or to the Pia matter. These regions may be affixed to the armature by means of a chamfered channel travelling the circumference of the openings which accommodate the proximal and distal ends of a severed spinal cord, along with an appropriate adhesive. These channels should be oriented parallel to the spinal cord.

The armature must have a suturable region travelling the circumference of the modular power supply port (8). This region is oriented perpendicularly to suturable region (6), and should travel the circumference of the region which allows for attachment of the modular power supply. This suturable region (8) is intended to affix the Dura matter of the spinal cord around the circumference of the modular power supply attachment region. By doing so, the modular power supply (11), rests outside of the tissues sheathing the spinal cord, thereby allowing modular power supplies to be interchanged (post-operatively) without having to make an incision into the sheaths of the spinal cord.

The armature must have ports to connect the power leads (4) leaving the inner sheath to the modular power supply (11). The armature, lastly, must have a means of securely attaching the modular power supply (11).

The modular power supply (11) of this for this apparatus is any device that can fit in the attachment area of the armature and provide an appropriate power supply to the power leads (4) of the apparatus. The coupling between the device and the modular power supply should take place outside the Dura matter of the spinal cord, into which the main body of the biocompatible armature is sutured. This should be affected by having the coupling area protrude dorsally from the main body of the device. This protrusion should be continuous with the biocompatible armature. This protrusion should have internally facing chamfers and be surrounded by a cuff of material capable of being sutured (8). The junction between the main device and the modular power supply can affected by any means capable of attaching the power supply adequately and that does not impede biocompatibility or implantability. The method of attaching the modular power supply pictured in FIG. 1 is a pressure fitting, which would allow the modular power supply to be pushed into position and firmly held by the internal chamfer of the modular power supply attachment region. The power supply of this device is should be modular to allow for upgrades to the power supply system over the life of the implant without having to remove the main body of the device.

The preferred method of powering this device would be a kinetic charger/rechargeable battery apparatus to generate power by means of body movement. Other methods that can be utilized are an internal induction coil/rechargeable battery system that would be charged periodically with an external induction coil/rechargeable battery system linked to a generator (solar/kinetic), or to a stationary power source. These methods are preferable power solutions as they would provide unimpeded and (largely) self generated electrical power. Other modular devices are acceptable so long as they provide an appropriate power supply, attach to the armature, and allow for uninterrupted power delivery.

For this device to be implemented surgically, the region to be repaired must be prepped for the implant. Preparation of the area requires that the damaged ends of the spinal cord must be cut so that they provide a flat plain perpendicular to the length of the cord. The vertebrae in the region to be repaired must also be immobilized (temporarily or permanently, depending on consultation with a surgeon and the patients activity level) using pre existing vertebral fusion/immobilization techniques. It should be implanted using appropriate microsurgery techniques.

Implementation of this device would be most favorable when coupled with physical therapy including biofeedback, exercise, and external stimulation of muscle groups. Any post operative therapies should be discussed with a physician.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### CITATIONS

(1) Gasson, Mark et. al. “Invasive neural prosthesis for neural signal detection and nerve stimulation.” International Journal of Adaptive Control and Signal Processing. 19.5 (2004): 365-375.
(2) Ibid.
(3) WO/2002/087683 (2002 Jul. 11) Ehud Cohen, Actuation and control of limbs through motor nerve stimulation

### US REFERENCES

5092332 (1992 Mar. 3) Philip Lee, Steroid eluting cuff electrode for peripheral nerve stimulation
4541432 (1985 Sep. 17) Pedro Molina-Negro, Electric nerve stimulator device
20080071321 (2008 Mar. 20) Joseph W. Boggs II, Systems and methods of neuromodulation stimulation for the restoration of sexual function
4969468 (1990 Nov. 13) Charles L. Byers, Electrode array for use in connection with a living body and method of manufacture
7292890 (2007 Nov. 6) Todd K. Whitehurst, Vagus nerve stimulation via unidirectional propagation of action potentials
20080228240 (2008 Sep. 16) David J. Edell, Long term bi-directional axon-electronic communication system

### FOREIGN REFERENCES

WO/2002/087683 (2002 Jul. 11) Ehud Cohen, Actuation and control of limbs through motor nerve stimulation
WO/2007/109228 (2007 Sep. 27) Javed Kahn, Apparatus for microarray binding sensors having biological probe materials using carbon nanotube transistors

### OTHER REFERENCES

Anthony, Catherine Parker (1975). “Textbook of Anatomy and Physiology” 9^{th} edition.
C.V. Mosby Company
Gray, Henry (1930). “Anatomy of the Human Body” 22^{nd} edition.
Philadelphia: Lea and Febiger
Martini, Frederic H. (2009). “Human Anatomy” 6^{th} edition.
San Francisco: Pearson Education
Silverthorn, Dee Unglaub (2007). “Human Physiology: An integrated approach” 4^{th} edition.
San Francisco: Pearson Cummings

1. This device will provide bi-directional nerve signal detection and stimulation across proximal and distal ends of a severed spinal cord.
Nerve signal detection and stimulation will be achieved through the use of arrays of carbon nanotube micro electrodes, one with a detection/stimulation surface facing the proximal end and one facing the distal end of a severed spinal cord.
Nanotube electrodes will occur in stimulator/sensor partner pairs on any one array with a mirrored pair of the microelectrodes on the oppositely oriented.
Nanotube electrode density will be at least 500 electrodes per square centimeter.
Signal relay will be achieved through a circuit which detects incoming signals from one partnered electrode pair on one array surface, amplifies it, and conducts it across to a mirrored pair on the opposite array.

2. This device will not impede flow of cerebral-spinal fluid through and around the spinal cord.
A channel or duct will allow the unimpeded flow of cerebral-spinal fluid through the center of this device.
Channels in the outer sheath of this device will allow unimpeded flow of cerebral-spinal fluid around the spinal cord.
Suture-able sections of this device will allow it to be secured in place within a body without impeded cerebral-spinal fluid flow.

3. This device is capable of accepting various modular power systems.
This device may be powered by any apparatus that can supply an appropriate power source while not impeding biocompatibility and implant-ability.

4. Data processing of this device is self contained.
This device will provide no external data output or receive any signal modulation control.

5. The cross-sectional shape of this device will be dependent upon the region of the spinal cord into which it would be implanted.
For each cross-sectional shape of this device, various sizes will account for the varying dimensions of the spinal cord of recipients at any specific level of the spinal cord.
